(19) United States
(12) Patent Application Publication
(10) Pub. No.: US 2008/0221115 A1
(43) Pub. Date: Sep. 11, 2008

(54) Use of long-chain alcohol derivatives for the treatment of alopecia areata

(76) Inventor: Liat Hayardeny-Nisimov, Tel Aviv (IL)

Correspondence Address: COOPER & DUNHAM, LLP, 1185 AVENUE OF THE AMERICAS, NEW YORK, NY, 10036, US

(21) Appl. No.: 12/072,304

(22) Filed: Feb. 25, 2008

Related U.S. Application Data
(60) Provisional application No. 60/903,784, filed on Feb. 26, 2007.

(51) Int. Cl. A61K 31/495; A61K 31/216; A61P 17/14

(52) U.S. Cl. 514/252.12; 514/529

(57) ABSTRACT

Disclosed is a method of treating alopecia greata using a compound having the formula:

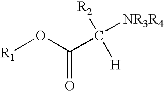

wherein R₁ is C₁₀-C₂₄ alkenyl; R₂ is H, C₁-C₆ alkyl, aryl, or aralkyl, where any aryl moiety may be unsubstituted or substituted by nitro, cyano, halo, hydroxyl, NR₆R₇, or CR₈R₈NR₆R₇, where R₆, R₇, and R₈ each independently is H or C₁-C₆ alkyl; and R₃ and R₄ each independently is H or C₁-C₆ alkyl, or R₃ and R₄ together with the nitrogen atom to which they are attached form a 5-7 membered heterocyclic saturated ring optionally containing an additional N or O, which is unsubstituted or substituted by C₁-C₆ alkyl, or an enantiomer or a pharmaceutically acceptable salt of the compound.

The application claims benefit of U.S. Provisional Application No. 60/903,784, filed Feb. 26, 2007, the contents of which are hereby incorporated by reference.

Throughout this application various publications, published patent applications, and patents are referenced. The disclosures of these documents in their entireties are hereby incorporated by reference into this application in order to more fully describe the state of the art to which this invention pertains.

### BACKGROUND OF THE INVENTION

Alopecia greata is a disease characterized by a localized area of complete hair loss (Springer, et al. “Common Hair Loss Disorders” American Family Physician 2003 68(1) 93-102). When the disease progresses to the point that all of the hair on a patient's scalp is lost, the disease is called alopecia greata totalis. When all of the hair on a patient's body is lost, the disease is called alopecia greata universalis. Alopecia greata may affect both men and women of all ages. The psychological effects due to loss of self-image due to hair loss may be great.

Topical immunomodulators are emerging as the therapy of choice for alopecia greata (J Postgrad Med. 2004 50(2) 131-9). Among this class of agents are corticosteroids either in topical, oral, and preferably intralesional administration forms (American Family Physician 2003 68(1) 99). Other immunomodulators include contact sensitizers, which potentially cause severe side effects (ibid).

Other therapies for alopecia greata include minoxidil, psoralen plus ultraviolet A and anthralin, but success rates vary (ibid). The need remains for an effective non-steroidal treatment of alopecia greata.

### SUMMARY OF THE INVENTION

The invention provides a method of treating a subject afflicted with alopecia greata comprising administering to the subject a compound having the formula:

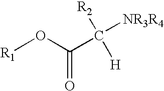

wherein R₁ is C₁₀-C₂₄ alkenyl; R₂ is H, C₁-C₆ alkyl, aryl, or aralkyl, where any aryl moiety may be unsubstituted or substituted by nitro, cyano, halo, hydroxyl, NR₆R₇, or CR₈R₈NR₆R₇, where R₆, R₇, and R₈ each independently is H or C₁-C₆ alkyl; and R₃ and R₄ each independently is H or C₁-C₆ alkyl, or R₃ and R₄ together with the nitrogen atom to which they are attached form a 5-7 membered heterocyclic saturated ring optionally containing an additional N or O, which is unsubstituted or substituted by C₁-C₆ alkyl, or an enantiomer or a pharmaceutically acceptable salt of the compound, in an amount effective to treat the subject.

In an embodiment of the method R₁ is C₁₆-C₂₀; R₂ is aryl, or aralkyl; R₃ and R₄ are each H, or R₃ and R₄ together with the nitrogen atom to which they are attached form a 5-7 membered heterocyclic saturated ring optionally containing an additional N or O, which is unsubstituted or substituted by C₁-C₆ alkyl, or an enantiomer or pharmaceutically acceptable salt of the compound.

The invention also provides a pharmaceutical composition for the treatment of alopecia greata comprising a pharmaceutically acceptable carrier and a compound of the formula:

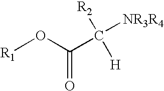

wherein R₁ is C₁₀-C₂₄ alkenyl; R₂ is H, C₁-C₆ alkyl, aryl, or aralkyl, where any aryl moiety may be unsubstituted or substituted by nitro, cyano, halo, hydroxyl, NR₆R₇, or CR₈R₈NR₆R₇, where R₆, R₇, and R₈ each independently is H or C₁-C₆ alkyl; and R₃ and R₄ each independently is H or C₁-C₆ alkyl, or R₃ and R₄ together with the nitrogen atom to which they are attached form a 5-7 membered heterocyclic saturated ring optionally containing an additional N or O, which is unsubstituted or substituted by C₁-C₆ alkyl, or an enantiomer or a pharmaceutically acceptable salt of the compound, in an amount effective to treat the subject.

The invention also provides the use of a compound having the formula:

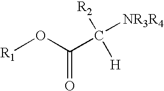

wherein R₁ is C₁₀-C₂₄ alkenyl; R₂ is H, C₁-C₆ alkyl, aryl, or aralkyl, where any aryl moiety may be unsubstituted or substituted by nitro, cyano, halo, hydroxyl, NR₆R₇, or CR₈R₈NR₆R₇, where R₆, R₇, and R₈ each independently is H or C₁-C₆ alkyl; and R₃ and R₄ each independently is H or C₁-C₆ alkyl, or R₃ and R₄ together with the nitrogen atom to which they are attached form a 5-7 membered heterocyclic saturated ring optionally containing an additional N or O, which is unsubstituted or substituted by C₁-C₆ alkyl, or an enantiomer or a pharmaceutically acceptable salt of the compound, in an amount effective to treat the subject, or of an enantiomer of the compound, or of a pharmaceutically acceptable salt of the compound, for the preparation of a medicament for the treatment of alopecia greata in an afflicted subject.

### DETAILED DESCRIPTION OF THE INVENTION

The invention provides a method of treating a subject afflicted with alopecia greata comprising administering to the subject a compound having the formula:

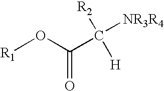

wherein R₁ is C₁₀-C₂₄ alkenyl; R₂ is H, C₁-C₆ alkyl, aryl, or aralkyl, where any aryl moiety may be unsubstituted or substituted by nitro, cyano, halo, hydroxyl, NR₆R₇, or CR₈R₈NR₆R₇, where R₆, R₇, and R₈ each independently is H or C₁-C₆ alkyl; and R₃ and R₄ each independently is H or C₁-C₆ alkyl, or R₃ and R₄ together with the nitrogen atom to which they are attached form a 5-7 membered heterocyclic saturated ring optionally containing an additional N or O, which is unsubstituted or substituted by C₁-C₆ alkyl, or an enantiomer or a pharmaceutically acceptable salt of the compound, in an amount effective to treat the subject.

In an embodiment of the method, R₁ is C₁₆-C₂₀; R₂ is aryl, or aralkyl; R₃ and R₄ are each H, or R₃ and R₄ together with the nitrogen atom to which they are attached form a 5-7 membered heterocyclic saturated ring optionally containing an additional N or O, which is unsubstituted or substituted by C₁-C₆ alkyl, or an enantiomer or pharmaceutically acceptable salt of the compound.

In another embodiment of the method R₁ is C₁₈ alkenyl.

In another embodiment of the method R₁ is cis-9-octadecenyl.

In another embodiment of the method R₂ is aryl, which is unsubstituted or substituted by nitro, cyano, halo, hydroxyl, NR₆R₇, or CR₈R₈NR₆R₇.

In another embodiment of the method R₂ is phenyl.

In another embodiment of the method R₂ is H.

In another embodiment of the method R₃ and R₄ are each H.

In another embodiment of the method R₃ and R₄ together with the nitrogen atom to which they are attached form a 5-7 membered heterocyclic saturated ring containing an additional N or O, which is unsubstituted or substituted by C₁-C₆ alkyl.

In an embodiment of method of R₃ and R₄ together with the nitrogen atom to which they are attached form a piperazinyl ring.

In another embodiment of the method the compound is amino-phenyl-acetic acid octadec-(Z)-9-enyl ester, or a pharmaceutically acceptable salt thereof.

In another embodiment of the method the compound is amino-phenyl-acetic acid octadec-(Z)-9-enyl ester HCl salt.

In another embodiment of the method the compound is enantioenriched or enantiopure(R)-amino-phenyl-acetic acid octadec-9-(Z)-enyl ester or a pharmaceutically acceptable salt thereof.

In another embodiment of the method the compound is enantioenriched or enantiopure(R)-amino-phenyl-acetic acid octadec-9-(Z)-enyl ester HCl salt.

In another embodiment of the method the compound is enantioenriched or enantiopure(S)-amino-phenyl-acetic acid octadec-9-(Z)-enyl ester or a pharmaceutically acceptable salt thereof.

In another embodiment of the method the compound is enantioenriched or enantiopure(S)-amino-phenyl-acetic acid octadec-9-(Z)-enyl ester HCl salt.

In another embodiment of the method the compound is piperazin-1-yl acetic acid octadec-(Z)-9-enyl ester or a pharmaceutically acceptable salt thereof.

In another embodiment of the method the compound is piperazin-1-yl acetic acid octadec-(Z)-9-enyl ester bitartrate salt.

In another embodiment of the method the compound is administered by topical administration.

In another embodiment of the method of the subject is human.

In another embodiment of the method the amount of the compound is effective to reduce hair loss in the subject.

The invention also provides a pharmaceutical composition for the treatment of alopecia greata comprising a pharmaceutically acceptable carrier and a compound of the formula:

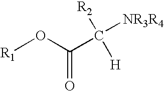

wherein R₁ is C₁₀-C₂₄ alkenyl; R₂ is H, C₁-C₆ alkyl, aryl, or aralkyl, where any aryl moiety may be unsubstituted or substituted by nitro, cyano, halo, hydroxyl, NR₆R₇, or CR₈R₈NR₆R₇, where R₆, R₇, and R₈ each independently is H or C₁-C₆ alkyl; and R₃ and R₄ each independently is H or C₁-C₆ alkyl, or R₃ and R₄ together with the nitrogen atom to which they are attached form a 5-7 membered heterocyclic saturated ring optionally containing an additional N or O, which is unsubstituted or substituted by C₁-C₆ alkyl, or an enantiomer or a pharmaceutically acceptable salt of the compound, in an amount effective to treat the subject.

The invention also provides the use of a compound having the formula:

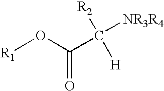

wherein R₁ is C₁₀-C₂₄ alkenyl; R₂ is H, C₁-C₆ alkyl, aryl, or aralkyl, where any aryl moiety may be unsubstituted or substituted by nitro, cyano, halo, hydroxyl, NR₆R₇, or CR₈R₈NR₆R₇, where R₆, R₇, and R₈ each independently is H or C₁-C₆ alkyl; and R₃ and R₄ each independently is H or C₁-C₆ alkyl, or R₃ and R₄ together with the nitrogen atom to which they are attached form a 5-7 membered heterocyclic saturated ring optionally containing an additional N or O, which is unsubstituted or substituted by C₁-C₆ alkyl, or an enantiomer or a pharmaceutically acceptable salt of the compound, in an amount effective to treat the subject, or of an enantiomer of the compound, or of a pharmaceutically acceptable salt of the compound, for the preparation of a medicament for the treatment of alopecia greata in an afflicted subject.

“Enantioenriched compound” or “enantiomerically enriched compound” as used here means a composition of a chiral substance whose enantiomeric ratio is greater than 50:50 but less than 100:0 of the specified enantiomer. (See IUPAC Compendium of Chemical Terminology, “Goldbook”, Second Edition, 1997).

“Enantiopure compound” or “enantiomerically pure compound” as used herein means a composition containing molecules all having the same chirality sense (within the limits of detection). (See IUPAC Compendium of Chemical Terminology, “Goldbook”, Second Edition, 1997).

“Racemic mixture”, “racemic composition”, “racemic”, “racemate” and “(±)” terminology are used interchangeably herein.

Certain embodiments of the disclosed compounds can contain a basic functional group, such as amino or alkylamino, and are thus capable of forming pharmaceutically acceptable salts with pharmaceutically acceptable acids, or contain an acidic functional group and are thus capable of forming pharmaceutically acceptable salts with bases. The instant compounds may be in a salt form. As used herein, a “salt” is salt of the instant compounds which has been modified by making acid or base salts of the compounds. In the case of compounds used for treatment of cancer, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

Enantioselective preparation of esters of phenylglycine is a challenging endeavor. (Clark et al., 1976, Journal of the Chemical Society: Perkin Transactions 1, 5:471-474) Amino acids can tautomerize and interconvert between their keto and enol tautomers; however in acidic or aqueous media, most will prefer the keto tautomer, making resolution of enantiomers possible. As most commonly encountered, tautomerization results in the formal migration of a hydrogen atom or proton, accompanied by a switch of a single bond and adjacent double bond. In solutions where tautomerization is possible, a chemical equilibrium of the tautomers will be reached. The exact ratio of the tautomers depends on several factors, including temperature, solvent, and pH. (see Organic Chemistry, McMurry, 2003) In the case of phenylglycine however, the phenyl ring stabilizes the enol form through double bond conjugation, giving rise to an equilibrium of both tautomers. Formation of the enol tautomer, and thus formation of an olefinic bond removes the previous chirality of the amino acid, and results in racemization of products. The stabilization of this achiral enol tautomer, makes enantioselective preparation of phenylglycine derivatives difficult; enantioselective preparation of amino-phenyl-acetic acid octadec-9-(Z)-enyl ester has not been reported prior to this invention.

Within the context of the invention, the term “isolated” means absent of another compound, in particular, absent of another enantiomer, as determined by standard currently available methods of analysis.

As used herein, the term “effective amount” refers to the quantity of a component that is sufficient to yield a desired therapeutic response without undue adverse side effects (such as toxicity, irritation, or allergic response) commensurate with a reasonable benefit/risk ratio when used in the manner of this invention. For example, an amount effective to inhibit or reverse symptoms of inflammation The specific effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

The esters of the present invention are in general crystalline, non-hygroscopic and water-soluble and are more easily purified and formulated for oral and parenteral formulation than the starting saturated or cis-unsaturated alcohols.

It should be noted that for the preparation of the esters of the invention wherein RI is a cis-alkenyl group, the starting cis-unsaturated alcohol such as oleyl alcohol, may be used in a substantially pure cis-unsaturated form meaning that the reagent contains at least about 80% of the cis-form. For example, the commercial oleyl alcohol is about 85% pure and most of the impurity consists of the trans analog (elaidyl alcohol).

As used herein, “alkyl” is intended to include both branched and straight-chain saturated aliphatic hydrocarbon groups having the specified number of carbon atoms. Thus, C₁-Cₙ as in “C₁-Cₙ alkyl” is defined to include groups having 1, 2, . . . , n−1 or n carbons in a linear or branched arrangement. As used herein, “alkyl” means C₁-Cₙ, and is defined to include groups having 1, 2, 3, 4, 5, 6 etc. carbons in a linear or branched arrangement, and specifically includes methyl, ethyl, propyl, butyl, pentyl, hexyl, and so on. “Alkyl” in regard to any of R¹ through R¹² as used here is C₁-Cₙ. “Alkoxy” represents an alkyl group of indicated number of carbon atoms attached through an oxygen bridge.

The term “alkyl” as used in the terms “-alkyl-OH”, “—NH-alkyl”, “-alkyl-(NH₂)”, “-alkyl-C(O) (OH”, and “—O-alkyl” are C₁-Cₙ alkyl as defined above, i.e. they include groups having 1, 2, 3, 4, 5, or n carbons in a linear or branched arrangement. For example methyl, ethyl, propyl, butyl, pentyl, or hexyl in a linear or branched arrangement.

The term “alkyl” as used in the term “—N(alkyl)₂” means C₁-Cₙ alkyl as defined above, i.e. they include groups having 1, 2, 3, 4, 5, or n carbons in a linear or branched arrangement. However, the two alkyl groups of “—N(alkyl)₂” need not necessarily be the same type of alkyl group. For example one alkyl may be chosen from the group methyl, ethyl, propyl, butyl, pentyl, or hexyl in a linear or branched arrangement and the other alkyl may be independently chosen from the group methyl, ethyl, propyl, butyl, pentyl, or hexyl.

The term “cycloalkyl” shall mean cyclic rings of alkanes of three to eight total carbon atoms, or any number within this range (i.e., cyclopropyl, cyclobutyl, cyclopentyl, cyclohexyl, cycloheptyl or cyclooctyl).

If no number of carbon atoms is specified, the term “alkenyl” refers to a non-aromatic hydrocarbon radical, straight or branched, containing at least 1 carbon to carbon double bond, and up to the maximum possible number of non-aromatic carbon-carbon double bonds may be present. For example, “C₂-C₆ alkenyl” means an alkenyl radical having 2, 3, 4, 5, or 6 carbon atoms, and at least 1 carbon-carbon double bond, and up to, for example, 5 carbon-carbon double bonds in the case of a C₆ alkenyl. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated. “Alkenyl” with regard to R¹ through R¹² as used here is C₂-Cₙ.

The term “cycloalkenyl” shall mean cyclic rings of 3 to 10 carbon atoms and at least 1 carbon to carbon double bond (i.e., cycloprenpyl, cyclobutenyl, cyclopenentyl, cyclohexenyl, cycloheptenyl or cycloocentyl).

As used herein, “aryl” is intended to mean any stable monocyclic or bicyclic carbon ring of up to 10 atoms in each ring, wherein at least one ring is aromatic. Examples of such aryl elements include phenyl, naphthyl, tetrahydro-naphthyl, indanyl, biphenyl, phenanthryl, anthryl or acenaphthyl. In cases where the aryl substituent is bicyclic and one ring is non-aromatic, it is understood that attachment is via the aromatic ring. The substituted aryls included in this invention include substitution at any suitable position with amines, substituted amines, alkylamines, hydroxys and alkylhydroxys, wherein the “alkyl” portion of the alkylamines and alkylhydroxys is a C₂-Cₙ alkyl as defined hereinabove. The substituted amines may be substituted with alkyl, alkenyl, alkynl, or aryl groups as hereinabove defined.

The term “heteroaryl”, as used herein, represents a stable monocyclic or bicyclic ring of up to 10 atoms in each ring, wherein at least one ring is aromatic and contains from 1 to 4 heteroatoms selected from the group consisting of O, N and S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The alkyl, alkenyl, alkynyl, cycloalkyl, aryl, heteroaryl and heterocyclyl substituents may be unsubstituted or unsubstituted, unless specifically defined otherwise. For example, a (C₁-C₆) alkyl may be substituted with one or more substituents selected from OH, oxo, halogen, alkoxy, dialkylamino, or heterocyclyl, such as morpholinyl, piperidinyl, and so on.

In the compounds of the present invention, alkyl, alkenyl, alkynyl, cycloalkyl, cycloalkenyl, heterocyclyl and heteroaryl groups can be further substituted by replacing one or more hydrogen atoms be alternative non-hydrogen groups. These include, but are not limited to, halo, hydroxy, mercapto, amino, carboxy, cyano and carbamoyl.

The term “substituted” shall be deemed to include multiple degrees of substitution by a named substitutent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

The compounds can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone but are generally mixed with a pharmaceutically acceptable carrier. This carrier can be a solid or liquid, and the type of carrier is generally chosen based on the type of administration being used. In one embodiment the carrier can be a monoclonal antibody. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Specific examples of pharmaceutical acceptable carriers and excipients that may be used to formulate oral dosage forms of the present invention are described in U.S. Pat. No. 3,903,297 to Robert, issued Sep. 2, 1975. Techniques and compositions for making dosage forms useful in the present invention are described-in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers Therapeutic Applications: Drugs and the Pharmaceutical Sciences, Vol. 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The pharmaceutical composition provided by the present invention may be in solid, semisolid or liquid form and may further include pharmaceutically acceptable fillers, carriers or diluents, and other inert ingredients and excipients.

The composition can be administered by any suitable route such as, but not limited to, oral, topical, or parenteral e.g. by injection through subcutaneous, intravenous, intramuscular, or any other suitable route. Since many of the compounds are oily, they are preferably administered parenterally, more preferably subcutaneously. If given continuously, the compounds of the present invention are each typically administered by 1-4 injections per day or by continuous subcutaneous infusions, for example, using a mini-pump. The dosage will depend on the state of the patient and severity of the disease and will be determined as deemed appropriate by the practitioner.

For parenteral administration, the compounds may be formulated by mixing the compound at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. Generally, the formulations are prepared by contacting the compound uniformly and intimately with liquid carriers or finely divided solid carriers or both. Then, if necessary, the product is shaped into the desired formulation. Preferably, the carrier is a parenteral carrier, more preferably a solution that is isotonic with the blood of the recipient. Examples of such carrier vehicles include water, saline, Ringer's solution, and dextrose solution. Non-aqueous vehicles such as fixed oils can be also useful, as well as liposomes. These preparations can be made by conventional methods known to those skilled in the art, for example as described in “Remington's Pharmaceutical Science”, A. R. Gennaro, ed., 17th edition, 1985, Mack Publishing Company, Easton, Pa., USA.

### EXPERIMENTAL DETAILS

### Example 1

### Preparation of Compounds

The oleyl alcohol derivatives amino-phenyl-acetic acid octadec-(Z)-9-enyl ester HCl and piperazin-1-yl acetic acid octadec-(Z)-9-enyl ester bitartrate are disclosed in PCT application publication WO 2004/032824 (compounds 11 and 9, therein.) Both of these derivatives show positive results in a delayed-type hypersensitivity model in mice. Piperazin-1-yl acetic acid octadec-(Z)-9-enyl ester bitartrate showed positive results in an EAE model in rats.

The compounds being tested herein, amino-phenyl-acetic acid octadec-(Z)-9-enyl ester HCl (compound 1) and piperazin-1-yl acetic acid octadec-(Z)-9-enyl ester bitartrate (compound 2) were prepared based on the synthesis described in PCT International Application Publication No. WO 2004/032824, as described below.

### Preparation of amino-phenyl-acetic acid octadec-(Z)-9-enyl ester HCl (Compound 1)

To a solution of N-Boc phenylglycine (2.51 g, 10 mmol) in acetonitrile (50 ml), was added portionwise 1,1′-carbonyldiimidazole (3.24 g, 20 mmol). The solution was stirred at room temperature for 1 h, oleyl alcohol (3.15 ml, 2.68 g, 10 mmol) was added, and the reaction mixture further stirred for 2 h at room temperature. The solvent was evaporated, the residue was dissolved in ethyl acetate (150 ml), washed successively with 5% NaHCO3, 5% citric acid and water, dried on MgS04 and evaporated to dryness. The residue was dissolved in 1% HCl in ethyl acetate (100 ml), and the solution set aside for 6 h at room temperature, and evaporated to dryness. The residue thus obtained was crystallized from ether/n-hexane to give 1.6 g 3.65 mmol, 36.5%) of an off-white solid, mp 101-103° C.

### Preparation of piperazin-1-yl acetic acid octadec-(Z)-9-enyl ester bitartrate (Compound 2)

To a solution of piperazine (10.77 g, 125 mmol) in acetonitrile (200 ml) was added a solution of crude oleyl chloroacetate (prepared from 25 mmol chloroacetyl chloride) in acetonitrile (30 ml). The mixture was refluxed for 1.5 h, cooled to room temperature and evaporated to dryness. Water and ethyl acetate were added to the residue, and the phases were separated. The organic phase was washed with an equal volume of water, dried on sodium sulfate, and evaporated to dryness. The oily residue was dissolved in a small volume of methanol, and L-tartaric acid (7.50 g, 2 eqs.) in MeOH was added. The solution of the crude salt was evaporated to dryness, and the solid residue treated with acetonitrile, filtered, washed with acetonitrile, and then with acetone. The solid was dissolved in MeOH (400 ml), filtered and evaporated to dryness. The residue was treated with EtOAc, filtered, washed with EtOAc, then with acetone and dried to give the title compound as a white, non-hygroscopic powder, mp 130-132° C. (10.45 g, 15 mmol, 60%).

Enantiomers of amino-phenyl-acetic acid octadec-(Z)-9-enyl ester can be synthesized using the method described below, namely, by heating a mixture of oleyl alcohol and a stereoisomer of phenylglycine chloride hydrochloride in acetonitrile:

(S)-Amino-phenyl-acetic acid octadec-9-(Z)-enyl ester HCl Oleyl alcohol (13.4 g) was added to a stirred mixture of L-phenylglycine chloride hydrochloride (10.3 g) and acetonitrile (140 ml). The reaction mixture was refluxed for 3 hr, cooled to 2-4° C. and kept at this temperature for 2 hr. The precipitated solid was collected by filtration and crystallized from 9:1 acetone:ethanol mixture, and dried to give 8.1 g (37%), mp 83.9-84.5° C., 100% enantiomeric excess (ee).

(R)-Amino-phenyl-acetic acid octadec-9-(Z)-enyl ester HCl Oleyl alcohol (2.68 g) was added to a stirred mixture of D-phenylglycine chloride hydrochloride (2.06 g) and acetonitrile (60 ml). The reaction mixture was refluxed for ½ hr, cooled to 2-4° C. and kept at this temperature for 1 hr. The precipitated solid was collected by filtration and crystallized from acetone, and dried to give 1.9 g (43%), mp 85.5-86.5° C., 100% ee.

The active treatment compounds were formulated into pharmaceutical compositions by dissolving 100 mg of each active treatment compound in 1 ml of phosphate buffered saline (PBS).

A rodent model of alopecia greata is the C3H/H3J mice colony. (Sundberg, et al. “Aloplecia Areata in Aging C3H/HeJ Mice” The Journal of Investigative Dermatology 1994, 102(6) 847-856; McElwee et al., “Alopecia Areata Susceptibility in Rodent Models” JID Symposium Proceedings 2003, 8(2) 182-187) Alopecia greata can be induced in C3H/HeJ mice by grafting alopecia greata affected skin onto young C3H/HeJ mice. The young mice will develop the disease subsequent to grafting.

Lesional grafts from old C3H/HeJ mice were grafted onto young C3H/HeJ mice. On day 30-40 post-graft the mice were randomized into groups with approximately the same degree of alopecia greata pathology.

Group 1: negative control, phosphate buffered saline (PBS).

Group 2: amino-phenyl-acetic acid octadec-(Z)-9-enyl ester HCl 25 mg/ml (compound 1) in PBS.

Group 3: piperazin-1-yl acetic acid octadec-(Z)-9-enyl ester bitartrate (compound 2) 25 mg/ml in PBS.

Treatment was initiated on the day of randomization into groups. Mice were treated by providing 40 μl of solution per cm² lesion. The solution was evenly spread and was allowed to soak into the skin. Mice were observed twice weekly and the size of patches was measured and documented.

Treatment solutions were prepared freshly each day.

At the end of the study, skin biopsies were evaluated in a blinded manner. The hair amount and hair growth from biopsies from mice mustache, face, head, neck, back, and stomach were graded from 1 to 5. A rating of 1 represented no hair, and a rating of 5 represented full growth. These scores were calculated for each mouse and averaged, and the average for each group is shown below as “average score.”

In addition, the mice were graded based on their overall appearance on a scale of 1 to 5 for “overall score.”

Score 1=50% of mouse hairless.

Score 2=20-40% of mouse hairless.

Score 3=hairless patch covering up to 20% of mouse.

Score 4=thin hair, or local small hairless patch.

Score 5=full hair.

There were 7 mice in each of the test compound groups and 5 mice in the negative control group.

The average scores and overall scores for the test compounds and for the negative control are listed in table 1 below. The scoring occurred on day 93 from the beginning of the treatment.

| Group | Overall score | Average score |
|---|---|---|
| 1 | 2.7 | 2.48 |
| 2 | 3.5 | 3.11 |
| 3 | 3.5 | 2.91 |

As is evident from the example presented above, the compounds 1 and 2 were effective in treating the alopecia greata in C3H/HeJ mice.

### Example 2

Additional compounds having structural similarity to compounds 1 and 2 are prepared. Specifically, the compounds have formula:

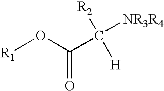

wherein R₁ is C₁₀-C₂₄ alkenyl; R₂ is H, C₁-C₆ alkyl, aryl, or aralkyl, where any aryl moiety may be unsubstituted or substituted by nitro, cyano, halo, hydroxyl, NR₆R₇, or CR₈R₈NR₆R₇, where R₆, R₇, and R₈ each independently is H or C₁-C₆ alkyl; and R₃ and R₄ each independently is H or C₁-C₆ alkyl, or R₃ and R₄ together with the nitrogen atom to which they are attached form a 5-7 membered heterocyclic saturated ring optionally containing an additional N or O, which is unsubstituted or substituted by C₁-C₆ alkyl, or an enantiomer or a pharmaceutically acceptable salt of the compound, in an amount effective to thereby treat the subject.

These additional compounds are each tested in a manner analogous to the testing performed with compounds 1 and 2 described herein. Each of the additional compounds is active in a manner analogous to the activity of compounds 1 and 2 described herein.

1. A method of treating a subject afflicted with alopecia greata comprising administering to the subject a compound having the formula: 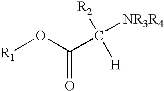
wherein
R₁ is C₁₀-C₂₄ alkenyl;
R₂ is H, C₁-C₆ alkyl, aryl, or aralkyl, where any aryl moiety may be unsubstituted or substituted by nitro, cyano, halo, hydroxyl, NR₆R₇, or CR₈R₈NR₆R₇, where R₆, R₇, and R₈ each independently is H or C₁-C₆ alkyl; and
R₃ and R₄ each independently is H or C₁-C₆ alkyl, or R₃ and R₄ together with the nitrogen atom to which they are attached form a 5-7 membered heterocyclic saturated ring optionally containing an additional N or O, which is unsubstituted or substituted by C₁-C₆ alkyl, or
an enantiomer or a pharmaceutically acceptable salt of the compound,
in an amount effective to treat the subject.

2. The method of claim 1, wherein
R₁ is C₁₆-C₂₀;
R₂ is aryl, or aralkyl;
R₃ and R₄ are each H, or R₃ and R₄ together with the nitrogen atom to which they are attached form a 5-7 membered heterocyclic saturated ring optionally containing an additional N or O, which is unsubstituted or substituted by C₁-C₆ alkyl,
or an enantiomer or pharmaceutically acceptable salt of the compound.

3. The method of claim 1, wherein R₁ is C₁₈ alkenyl.

4. The method of claim 3, wherein R₁ is cis-9-octadecenyl.

5. The method of claim 1, wherein R₂ is aryl, which is unsubstituted or substituted by nitro, cyano, halo, hydroxyl, NR₆R₇, or CR₈R₈NR₆R₇.

6. The method of claim 5, wherein R₂ is phenyl.

7. The method of any of claim 1, wherein R₂ is H.

8. The method of any of claim 1, wherein R₃ and R₄ are each H.

9. The method of claim 1, wherein R₃ and R₄ together with the nitrogen atom to which they are attached form a 5-7 membered heterocyclic saturated ring containing an additional N or O, which is unsubstituted or substituted by C₁-C₆ alkyl.

10. The method of claim 9 wherein R₃ and R₄ together with the nitrogen atom to which they are attached form a piperazinyl ring.

11. The method of claim 8 wherein the compound is amino-phenyl-acetic acid octadec-(Z)-9-enyl ester, or a pharmaceutically acceptable salt thereof.

12. The method of claim 11 wherein the compound is amino-phenyl-acetic acid octadec-(Z)-9-enyl ester HCl salt.

13. The method of claim 11 wherein the compound is enantioenriched or enantiopure(R)-amino-phenyl-acetic acid octadec-9-(Z)-enyl ester or a pharmaceutically acceptable salt thereof.

14. The method of claim 13 wherein the compound is enantioenriched or enantiopure(R)-amino-phenyl-acetic acid octadec-9-(Z)-enyl ester HCl salt.

15. The method of claim 11 wherein the compound is enantioenriched or enantiopure(S)-amino-phenyl-acetic acid octadec-9-(Z)-enyl ester or a pharmaceutically acceptable salt thereof.

16. The method of claim 15 wherein the compound is enantioenriched or enantiopure(S)-amino-phenyl-acetic acid octadec-9-(Z)-enyl ester HCl salt.

17. The method of claim 10 wherein the compound is piperazin-1-yl acetic acid octadec-(Z)-9-enyl ester or a pharmaceutically acceptable salt thereof.

18. The method of claim 17 wherein the compound is piperazin-1-yl acetic acid octadec-(Z)-9-enyl ester bitartrate salt.

19. The method of claim 1, wherein the compound is administered by topical administration.

20. The method of claim 1, wherein the subject is human.

21. The method of claim 1, wherein the amount of the compound is effective to reduce hair loss in the subject.

22. (canceled)

23. (canceled)
